(19) United States
(12) Patent Application Publication
(10) Pub. No.: US 2022/0249341 A1
(43) Pub. Date: Aug. 11, 2022

(54) CLEANSING COMPOSITION PROVIDING ENHANCED DEPOSITION OF SALICYLIC ACID FOR ACNE TREATMENT

(71) Applicant: L'OREAL, Paris (FR)

(72) Inventor: Siliu TAN, Westfield, NJ (US)

(73) Assignee: L'OREAL, Paris (FR)

(21) Appl. No.: 17/162,392

(22) Filed: Jan. 29, 2021

(51) Int. Cl. A61K 8/368; A61Q 19/10; A61K 8/73

(52) U.S. Cl. CPC A61K 8/368; A61Q 19/10; A61K 2800/5426; A61K 8/737; A61K 8/736

(57) ABSTRACT

A cosmetic composition for treating acne includes at least one acne treatment active that includes at least one beta hydroxy acid and an aqueous carrier comprising at least one cationic polymer. In some embodiments the cosmetic composition is a cleanser that comprises at least one surfactant. When the composition is applied to a keratinous tissue it demonstrates deposition and retention of the beta hydroxy acid on the keratinous tissue after rinsing with water in an amount representing about at least 60% and up to about 250% greater as compared with an amount deposited using a composition that lacks the at least one cationic polymer.

### FIELD OF THE INVENTION

The present disclosure is directed to a skin care composition, in particular, cosmetic cleansing composition with high efficacy to deliver actives for addressing acne.

### BACKGROUND OF THE INVENTION

Acne is a common skin disorder and is treated using a variety of different agents. Salicylic acid, a mild beta-hydroxy acid, known for facilitating sloughing of dead skin cells and other cellular debris, is often used in treating acne, as well as other skin conditions that include psoriasis, keratoses, and ichthyoses. Commercial forms of cosmetic products that include salicylic acid include peels, cleansers, and leave on treatment in the forms of lotions, creams, and make ups. A challenge with many of these product forms is that they delivery salicylic acid that is only transiently available on the skin, such as in cleansers and peels, or that is in relatively low amounts, thus precluding sustained effectiveness on the skin for addressing acne and other skin conditions that benefit from the agent.

Accordingly, there is a need for a composition that overcomes the shortcomings of the prior art and provides benefits that include the ability to cleanse skin and provide persistent delivery of a skin active, for example salicylic acid, after cleansing to enhance the treatment of skin with acne or that is prone to acne. The present invention provides such a composition.

### BRIEF SUMMARY OF THE INVENTION

In accordance with the various embodiments, a cosmetic composition is provided for cleansing the skin in a rinse-off formulation that provides a stable disposition of an acne treatment active, for example, beta hydroxy acid, on the skin that is resistant to removal by rinsing and/or use of a cleansing apparatus, such as a cleansing brush. In the various embodiments, the inventive composition includes at least one or a combination of cationic polymers. The retention of acne treatment active on the skin even beyond cleansing, in some embodiments with a cleansing brush, effectively extends the duration of the acne treatment active availability on the skin for addressing acne and other skin conditions that are responsive to acne treatment actives, in particular, salicylic acid and other beta hydroxy acids, thereby providing sustained treatment in an otherwise rinsible formulation. In some embodiments, the composition is formulated as a cleanser.

In some embodiments, the disclosure provides a cosmetic composition that includes at least acne treatment active comprising at least one beta hydroxy acid selected from -n-octanoylsalicylic acid, salicylate, sodium salicylate, willow extract, beta hydroxybutanoic acid, tropic acid, and trethocanic acid. The acne treatment active comprising at least one beta hydroxy acid is present in an aqueous carrier that includes one or more cationic polymers. In some embodiments the composition comprises at least one surfactant, or a combination of surfactants.

The composition when applied to a keratinous tissue demonstrates deposition and retention of the beta hydroxy acid on the keratinous tissue after rinsing with water in an amount representing about 60% to about 250% as compared with an amount deposited with a composition that lacks the one or more cationic polymers, wherein the deposition and enhanced salicylic acid retention may be demonstrated by measurement of color change resulting from complex formation between the acid and a metal salt, such as FeCl₃.

In various embodiments, the acne treatment active comprising at least one beta hydroxy acid is present in the composition in a range from about 0.1% to about 2%, by weight, based on the total weight of the composition. In some embodiments, the at least one beta hydroxy acid is salicylic acid.

In various embodiments, the composition has a pH that is in the range from about 3 to about 7, or from about 3 to about 5, or from about 3 to about 4, or from about 4 to about 5.

In some embodiments, the composition includes one or more salts, for example, the composition may include one or more salts selected from, but not limited to, sodium chloride, calcium chloride, potassium chloride, magnesium sulfate, magnesium carbonate, calcium carbonate, and zinc pyrrolidone carboxylic acid.

In various embodiments, the one or more salts, when present, are present in the composition in a range from about 0.01% to about 2%, by weight, based on the total weight of the composition.

In some embodiments, the at least one cationic polymer is a cationic nature-based polymer selected from polysaccharides. In some embodiments, the at least one cationic polymer is selected from chitosan, cationic guar, guar gum, guar hydroxypropyltrimonium chloride, hydroxypropyl guar, acacia senegal gum, Caesalpinia spinosa gum, agar, carob gum, carrageenan, carrageenan (lambda, iota kappa), cellulose gum, corn starch modified, dehydroxanthan gum, gellan gum, gum acacia, gum arabic, gum ghatti, gum karaya, gum tragacanth, hydroxypropyl methyl cellulose, lecithin, microcrystalline cellulose, pectin, potato starch modified, pullulan, sclerotium gum, sodium carboxymethyl starch, sorbitol/sebacic acid copolymer, xanthan gum, and mixtures thereof.

In some embodiments, the at least one nature based polymer is selected from chitosan and cationic guar. In some embodiments, the at least one nature based polymer includes only chitosan or only cationic guar. In some embodiments, the at least one nature based polymer includes a combination of chitosan with one or more of cationic guar or another cationic form of guar. In some embodiments, the at least one cationic polymer is a synthetic cationic polymer selected from the group consisting of polyquaterniums, cationic acrylic polymers, and combinations thereof. In some particular embodiments, the at least one cationic polymer is selected from the group consisting of chitosan, cationic guar, an amorphous functional acrylic polymer grafted onto a polyethylene backbone, polyquaternium-47, and combinations thereof.

In various embodiments, each of the at least one cationic polymer is present in an amount that ranges from about 0.1% to about 0.5% by weight.

In various embodiments, the composition includes at least one surfactant selected from the group consisting of nonionic surfactants, cationic surfactants, anionic surfactants, amphoteric surfactants, and combinations thereof. In some embodiments the composition includes a combination of amphoteric, nonionic, and anionic surfactants.

In some embodiments, the composition includes a combination of amphoteric, nonionic, and anionic surfactants comprising Coco Betaine, Glyceryl Stearate, PEG-150 Distearate, and Sodium Methyl Cocoyl Taurate.

In various embodiments, each of the at least one surfactant is present in an amount that ranges from about 0.1% to about 20% by weight.

In some embodiments, the composition includes one or more of other optional ingredients selected from the group consisting of skin care actives, humectants, conditioning agents, thickeners, viscosity adjusters, cooling agents, fillers, antimicrobials, preservatives, pH adjusters, chelating agents, and combinations thereof. In some embodiments, the composition includes a blend of humectants, antimicrobials, preservatives, actives, pH adjusters and chelating agents comprising C12-15 alkyl lactate and C12-15 alcohols, citric acid, trisodium ethylenediamine disuccinate, phenoxyethanol, caprylyl glycol, chlorphenesin, ammonium polyacryloyldimethyl taurate, propylene glycol and PEG-55 propylene glycol oleate, and kaolin.

In various embodiments, the disclosure provides a method for delivering to skin a cleansing composition capable of depositing salicylic acid for persistent retention on the skin after rinsing, the composition comprising: at least one beta hydroxy acid selected from -n-octanoylsalicylic acid, salicylate, sodium salicylate, willow extract, beta hydroxybutanoic acid, tropic acid, and trethocanic acid, and a aqueous carrier comprising: at least one cationic polymer, particularly a nature-based polymer selected from cationic polysaccharides; a blend of surfactants; and water; and, wherein the cosmetic composition is applied to skin that is prone to acne, followed by washing or scrubbing, optionally with a provided brush or other applicator, followed by rinsing.

Use of the cosmetic cleansing composition for treatment of skin conditions such as acne is characterized by deposition and retention on the skin after rinsing of the beta hydroxy acid, for example, salicylic acid, in an amount representing about 60% to about 250% as compared with an amount deposited with a composition that lacks the one or more cationic polymers, wherein the deposition and enhanced salicylic acid retention may be demonstrated by measurement of color change resulting from complex formation between the acid and a metal salt, such as FeCl₃.

It is to be understood that both the foregoing general description and the following detailed description are exemplary and explanatory only and are not restrictive of the invention.

### DETAILED DESCRIPTION OF THE INVENTION

The term “cleanser” as used herein can be any cosmetic composition utilized for application to a keratinous tissue for cleansing the skin, removal of make-up and the like.

The term “Nature-based” as used herein means and refers to cosmetically acceptable materials and components that are one or more of directly obtained from nature, are obtained from nature with minimal processing, and are derivatives of materials that are obtained from nature. Cosmetic compositions according to the instant disclosure are in some embodiments up to 99.11% natural.

The term “Skin” as used herein includes skin materials containing keratin such as facial and body skin, scalp, eyebrows, and lips.

The term “stable disposition of an acne treatment active” as used herein means deposition, for example, beta hydroxy acid, on the skin that is resistant to removal by rinsing and/or use of a cleansing apparatus, such as a cleansing brush.

The cosmetic composition, can comprise, consist of, or consist essentially of the essential elements and limitations of the invention described herein, as well as any additional or optional ingredients, components, or limitations described herein or otherwise useful.

The cosmetic composition may be formulated as a cleanser, as described herein, for cleansing the skin in a rinse-off formulation that provides a stable disposition of an acne treatment active, for example, beta hydroxy acid, on the skin that is resistant to removal by rinsing and/or use of a cleansing apparatus, such as a cleansing brush. The retention of salicylic acid on the skin even beyond cleansing with our without a cleansing brush effectively extends the duration of the salicylic acid availability on the skin for addressing acne and other skin conditions that are responsive to salicylic acid and other beta hydroxy acids, thereby providing sustained treatment in an otherwise rinsible formulation. In various embodiments, the inventive composition includes at least one or a combination of cationic polymers. In some embodiments the acne treatment active is salicylic acid.

The cosmetic composition overcomes shortcomings in the art pertinent to salicylic acid induced instability in cosmetic compositions. Unexpectedly, employing various embodiments of the composition as disclosed and as exemplified herein, the composition demonstrates deposition and retention of beta hydroxy acid, after rinsing, that may be shown by measurement of color change resulting from complex formation between the acid and a metal salt, such as FeCl₃. The deposition and enhanced salicylic acid retention was demonstrated herein in the in vitro tests by measurement of color change resulting from complex formation between the acid and a metal salt, such as FeCl₃. As shown in the examples, the composition, when applied to a keratinous tissue demonstrates deposition and retention of the beta hydroxy acid on the keratinous tissue after rinsing with water in an amount representing about 60% to about 250% as compared with an amount deposited with a composition that lacks the one or more cationic polymers, wherein the deposition and enhanced salicylic acid retention may be demonstrated by measurement of color change resulting from complex formation between the acid and a metal salt, such as FeCl₃. In vitro tests reported herein have confirmed deposition of salicylic acid on a pseudo skin material, and retention after rinsing, for example, through at least a first rinsing of about 30 seconds and removal of the composition using a brush apparatus.

Cosmetic Composition

In accordance with the various embodiments, the cosmetic composition is suitable for application to keratinous tissue for cleansing, particularly for addressing acne. In some embodiments, the cosmetic composition is free of, or substantially free of, or devoid of one or more of oils, silicones, and film formers. In other embodiments, the composition may comprise any one or more of ingredients comprising oils, silicone oils, silicone polymers, and film formers.

Beta Hydroxy Acid

In accordance with the various embodiments, the cosmetic composition comprises at least one acne treatment active comprising beta hydroxy acid. In some embodiments, the composition includes a beta hydroxy acid comprising salicylic acid.

In some particular embodiments according to the disclosure, the composition includes salicylic acid present in the cosmetic composition in an amount that ranges from about 0.2% to about 2% by weight, based on the total weight of the composition.

The term “beta-hydroxy acid” is understood to mean, according to the present invention, a carboxylic acid having a hydroxyl functional group and a carboxylic functional group separated by two carbon atoms. A beta hydroxy acid can be present in the cosmetic composition in the form of the free acid and/or in the form of one of its associated salts (salts with an organic base or an alkali metal, in particular), especially according to the final pH imposed on the composition.

The beta hydroxy acid is selected from one or more of salicylic acid and derivatives thereof (including 5-n-octanoylsalicylic acid, salicylate, sodium salicylate, and willow extract), capryloyl salicylic acid, beta hydroxybutanoic acid, tropic acid, and trethocanic acid.

The total amount of beta hydroxy acid includes, in some particular embodiments, from about 0.1% up to and not more than about 2% of beta hydroxy acid, by weight, based on the total weight of the cosmetic composition. In some embodiments, the cosmetic composition includes and up to and not more than about 2%, or about 1.9% of beta hydroxy acid. In some embodiments, the cosmetic composition includes from about 0.1% to about 1% of beta hydroxy acid. In some embodiments, the cosmetic composition includes and up to and not more than about 1% of beta hydroxy acid. In some embodiments, the cosmetic composition includes more than about 2% beta hydroxy acid.

In accordance with the various embodiments, the amount of beta-hydroxy acid present in the compositions is in the range from about 0.1% to about 2%, and in some embodiments from about 0.1% to about 1.5%, and in some embodiments from about 0.1% to about 1%, including increments and all ranges and subranges therein and there between, by weight, based on the total weight of the composition. One of ordinary skill in the art, however, will appreciate that other ranges are within the scope of the invention.

Thus, the at least one beta hydroxy acid is present in the cosmetic composition from about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, to about 2.0 weight percent, including increments and all ranges and subranges therein and there between.

Cationic Polymer

In accordance with the various embodiments, the cosmetic composition comprises at least one cationic polymer selected from nature-based polymers that are polysaccharides, and other natural (i.e., plant, animal, or bacterial based), synthetic, or modified cationic nature-based or synthetic polymers.

In some embodiments, the cationic polymer is a nature-based polymer. In general, a cationic nature-based polymer may be selected from cationic forms and cationic derivatives of polysaccharides isolated from algae, polysaccharides produced by microorganisms, and polysaccharides from higher plants, such as homogeneous polysaccharides.

In some embodiments, cationic nature-based polymer selected from polysaccharides may be chosen from polysaccharides that include chitosan, chitin, starches, alginates, celluloses, galactomannans such as guar gums, particularly cationic derivatives thereof, and combinations thereof. In some embodiments, the at least one cationic nature-based polymer selected from polysaccharides may be chosen from chitosan, chitosan derivatives, chitin, starch, starch derivatives, cellulose (for example, but not limited to, ethylcellulose, nitrocellulose, hemicellulose, and hemicellulose derivatives), alginates, including but not limited to, sodium alginate, and combinations thereof.

In some particular embodiments, each of the at least one cationic nature-based polymer selected from polysaccharides may be chosen from chitosan and galactomannans such as guar gums, particularly cationic derivatives thereof.

In other embodiments, cationic nature-based polymer selected from polysaccharides may be chosen from one or more of methylcelluloses, hydroxyalkylcelluloses, ethylhydroxyethylcelluloses and carboxymethylcelluloses, mannans, xylans, lignins, arabans, galacturonans, alginate-based compounds, chitin, glucuronoxylans, arabinoxylans, xyloglucans, glucomannans, fructosans such as inulin, pectic acids and pectins, arabinogalactans, agars, glycosaminoglycans, gum arabics, tragacanth gums, ghatti gums, karaya gums, locust bean gums, biopolysaccharide gums of microbial origin such as scleroglucan or xanthan gums, mucopolysaccharides, chondroitin sulfates, and mixtures thereof.

In accordance with the various embodiments, the amount of each of the at least one cationic nature-based polymer present in the compositions can range from about 0.01% to about 10%, or from about 0.01% to about 5%, or from about 0.01% to about 0.5%, or from about 0.1% to about 0.5%, or from about 0.1% to about 0.2%, or from about 0.1% to about 5% or from about 1% to about 3.5% or any suitable combination, sub-combination, range, or sub-range thereof by weight, based on the weight of the composition. One of ordinary skill in the art, however, will appreciate that other ranges are within the scope of the invention.

In accordance with the various embodiments, the total amount of cationic nature-based polymer that may be present in the composition can range from about 0.01% to about 20%, or from about 0.05% to about 10%, or from about 0.1% to about 5%, or from about 0.05% to about 1%, or from about 0.01% to about 0.5%, or from about 0.1% to about 0.5%, or from about 0.1% to about 0.2%, or about 0.2% or any suitable combination, sub-combination, range, or sub-range thereof by weight, based on the weight of the composition. One of ordinary skill in the art, however, will appreciate that other ranges are within the scope of the invention.

Thus, any one of the at least cationic nature-based polymer, when present, is present, by weight, based on the total weight of the composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, to about 10 weight percent, including increments and ranges therein and there between.

In some embodiments, the cationic polymer is not a nature-based polymer and may be selected from synthetic polymers. In some examples, the synthetic polymers are non-silicone based, and in some embodiments may be selected from the group consisting of polyquaterniums, that is polymers having quaternary ammonium centers in the polymer, for example, including polyquaternium-47 (1-Propanaminium, N,N,N-trimethyl-3-[(2-methyl-2-propenyl)amino]-, chloride, polymer with methyl 2-propenoate and 2-propenoic acid). Other non-limiting examples of such polyquaternium compounds may be selected from diallyidimethylammonium chloride/acrylic acid copolymers sold under the names MERQUAT 280 POLYMER or MERQUAT 280NP POLYMER or MERQUAT 281 POLYMER or MERQUAT 295 POLYMER, by the company Nalco (Lubrizol) (INCI name: Polyquaternium-22); the copolymer of methacrylamidopropyltrimonium chloride, of acrylic acid and or methyl acrylate, sold under the name MERQUAT 2001 POLYMER OR MERQUAT 2001N POLYMER by the company Nalco (Lubrizol) (INCI name: Polyquaternium-47); the acrylamide/dimethyldiallylammonium chloride/acrylic acid terpolymer sold under the name MERQUAT 3330DRY POLYMER or MERQUAT 3330PR POLYMER or MERQUAT 3331PR POLYMER or MERQUAT 3940 POLYMER or MERQUAT PLUS 3330 POLYMER OR MERQUAT PLUS 3331 POLYMER by the company Nalco (Lubrizol) (INCI name: Polyquaternium-39); an ampholytic terpolymer consisting of methacrylamidopropyl trimethyl ammonium chloride (MAPTAC), acrylamide and acrylic acid, sold under the name MERQUAT 2003PR POLYMER by the company Nalco (Lubrizol) (INCI name: Polyquaternium-53); Polyquaternium-30, Polyquaternium-35, Polyquaternium-45, Polyquaternium-50, Polyquaternium-54; Polyquaternium-57; Polyquaternium-63; Polyquaternium-74; Polyquaternium-76; Polyquaternium-86; Polyquaternium-89; Polyquaternium-95; Polyquaternium-98, Polyquaternium-104; Polyquaternium-111; Polyquaternium-112, and mixtures thereof. In some embodiments, the cationic polymer is a synthetic polymer selected from cationic acrylic polymers, for example, an amorphous functional acrylic polymer grafted onto a polyethylene backbone such as SYNTRAN™ 5330, which is a quaternary modified olefin grafted technology. More generally, such synthetic polymers selected from, but are not limited to, cationic polymers comprising polyacrylates such as those identified in the International Cosmetic Ingredient Dictionary and Handbook (9 th ed. 2002) such as, for example, polyacrylate-1, polyacrylate-2, polyacrylate-3, polyacrylate-4, polyacrylate-16, polyacrylate-17, polyacrylate-18, polyacrylate-19, polyacrylate-21, and mixtures thereof. Such (co)polymers, or similar (co)polymers, can be combined individually or with other (co)polymers in such a way to form suitable bimodal agents having both cationic and anionic functionalities.

In accordance with the various embodiments, the amount of cationic polymer is not a nature-based polymer and is selected from synthetic cationic polymers that may be present in the composition can range from about 0.01% to about 20%, or from about 0.05% to about 10%, or from about 0.1% to about 5%, or from about 0.5% to about 2%, or about 1%, or from about 0.05% to about 1%, or from about 0.01% to about 0.5%, or from about 0.1% to about 0.5%, or from about 0.1% to about 0.2% or any suitable combination, sub-combination, range, or sub-range thereof by weight, based on the weight of the composition.

Thus, any one of the at least synthetic cationic polymer, when present, is present, by weight, based on the total weight of the composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, to about 10 weight percent, including increments and ranges therein and there between.

In accordance with the various embodiments, the amount of each of the at least one cationic polymer which may be either nature-based or synthetic cationic polymer, alone or in combination with another cationic polymer, present in the compositions can range from about 0.01% to about 10%, or from about 0.01% to about 5%, or from about 0.01% to about 0.5%, or from about 0.1% to about 0.5%, or from about 0.1% to about 0.2%, or from about 0.1% to about 5% or from about 1% to about 3.5%, or from about 0.5% to about 2%, or about 1%, or any suitable combination, sub-combination, range, or sub-range thereof by weight, based on the weight of the composition. One of ordinary skill in the art, however, will appreciate that other ranges are within the scope of the invention.

Salt

In some embodiments, the composition may include one or more salts, for example, the composition may include one or more salts selected from sodium chloride, calcium chloride, potassium chloride, magnesium sulfate, magnesium carbonate, calcium carbonate, and zinc pyrrolidone carboxylic acid, and other suitable mineral salts of sodium, calcium, magnesium, potassium, zinc, and the like.

And according to such embodiments, the amount of each of the one or more salts, when present, is in a range from about 0.01% to about 5%, or from about 0.01% to about 1%, or from about 0.01% to about 0.5%, or from about 0.1% to about 0.5%, or from about 0.1% to about 0.2%, or about 0.2%, or any suitable combination, sub-combination, range, or sub-range thereof by weight, based on the weight of the composition. One of ordinary skill in the art, however, will appreciate that other ranges are within the scope of the invention.

Thus, any one of the added salts, when present, is present, by weight, based on the total weight of the composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, to about 5 weight percent, including increments and ranges therein and there between.

Surfactant

In accordance with the various embodiments, the cosmetic composition comprises at least one surfactant. In some embodiments, the cosmetic composition comprises more than one surfactant. The at least one surfactant may be an nonionic, cationic, anionic, or an amphoteric or zwitterionic surfactant. In some embodiments, the composition includes one or a combination of surfactants selected from the group consisting of amphoteric, nonionic, anionic surfactants and combinations thereof.

In some embodiments, the amphoteric surfactant may include coco betaine, the nonionic surfactant may include glyceryl stearate or PEG-150 distearate or a combination thereof, and the anionic surfactant may include sodium methyl cocoyl taurate. In some particular embodiments, the composition includes the combination of surfactants that comprise coco betaine, glyceryl stearate, PEG-150 distearate, and sodium methyl cocoyl taurate.

In some embodiments, the composition includes one or a combination of surfactants that include surfactants selected from the group consisting of amphoteric, nonionic, anionic surfactants and combinations thereof. In some embodiments, the amphoteric surfactant may include Cocamidopropyl Hydroxysultaine, the nonionic surfactants may include PEG-150 Pentaerythrityl Tetrastearate, and the anionic surfactants may include Sodium Lauroyl Sarcosinate or Sodium Methyl Cocoyl Taurate or a combination thereof. In some embodiments, the composition includes the combination of surfactants that comprise Cocamidopropyl Hydroxysultaine, PEG-150 Pentaerythrityl Tetrastearate, Sodium Lauroyl Sarcosinate, and Sodium Methyl Cocoyl Taurate.

In some embodiments, the composition includes one or a combination of surfactants that include surfactants selected from the group consisting of amphoteric, nonionic, anionic surfactants and combinations thereof. In some embodiments, the amphoteric surfactant may include Coco Betaine, the nonionic surfactants may include Decyl Glucoside or PEG-150 Pentaerythrityl Tetrastearate or PEG-6 Caprylic/Capric Glycerides or a combination thereof, and the anionic surfactants may include Sodium Laureth Sulfate. In some embodiments, the composition includes the combination of surfactants that comprise Coco Betaine, Decyl Glucoside, PEG-150 Pentaerythrityl Tetrastearate, PEG-6 Caprylic/Capric Glycerides and Sodium Laureth Sulfate.

More generally, in some embodiments, the at least one surfactant may be selected from the group consisting of cocamidopropyl hydroxysultaine, sodium lauroyl sarcosinate, sodium methyl cocoyl taurate (and) sodium chloride (and) coconut acid, PEG-150 distearate, PEG-150 pentaerythrityl tetrastearate and combinations thereof. In some particular embodiments, the composition comprises more than one or all of the surfactants selected from the group consisting of cocamidopropyl hydroxysultaine, sodium lauroyl sarcosinate, sodium methyl cocoyl taurate (and) sodium chloride (and) coconut acid, PEG-150 distearate, PEG-150 pentaerythrityl tetrastearate and combinations thereof.

In some embodiments, the at least one surfactant may be selected from the group consisting of polyoxyethylene sorbitan monolaureate, laureth-23, polyoxyethylated octyl phenol, 3-((3-cholamidopropyl) dimethylammonio)-1 propane sulfonate, sodium dilauramidoglutamide lysine, cholate, deoxycholate, sodium dodecylsulfate, TWEEN-80, and combinations thereof. The at least one surfactant may exclude esters. The at least one surfactant may be present in the composition in a range from about 1% to about 5%, based on the weight of the composition.

In some embodiments, the at least one surfactant may be selected from the group consisting of surfactants with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di-C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate and combinations thereof.

In some embodiments, the at least one surfactant may be selected from the group consisting of PEG-100 Stearate; PEG-20 Stearate and other esters of Poly(Ethylene Glycol); Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers sodium C14-16 olefin sulfonate, disodium laureth sulfosuccinate (and) sodium lauryl sulfoacetate, sodium lauroyl sarcosinate, sodium methyl cocoyl taurate, sodium methyl lauroyl taurate, sodium cocoyl alaninate, sodium cocoyl isethionate, cocamidopropyl hydroxysultaine, cocamidopropyl betaine, coco-betaine, sodium cocoyl glycinate, sodium lauroyl glutamate, potassium cocoyl glycinate, decyl glucoside, lauryl glucoside, and combinations thereof.

In various embodiments, each of the at least one surfactant may be present in an amount in the range of from about 0.1% to about 20%, and the total amount of surfactant may be present in a range from about 0.1% to about 40% or more. In some embodiments, each of the at least one surfactant, when present, is present in an amount that is at least 0.1%, or about 1%, to about 20%, or up to 20%, or about 0.1% to about 5%, or to about 3% or to about 8%, or from about 5% to about 10%, or from about 7% to about 15%, or from about 10% to about 30%, or about 30% to about 50%, or any value, range, or sub-range therebetween by weight, based on the weight of the composition.

Thus, in various embodiments, each of the at least one surfactant may be present in a composition according to the disclosure from 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 120, 21, 22, 23, 24, 25, 26, 27, 28, 29, 30, 3, 32, 33, 34, 35, 36, 37, 38, 39, 40, 41, 42, 43, 44, 45, 46, 47, 48, 49 to about 50 percent, including increments and ranges therein and there between.

Water

In accordance with the various embodiments, the cosmetic composition includes water.

In various embodiments, the cosmetic composition comprises from about 5% to about 90% water, and in some embodiments from about 20% to about 85% water, and in some embodiments from about 50% to about 85% water, and in some embodiments from about 60% to about 80% water, and in some embodiments from about 65% to about 75%, water, including increments and all ranges and subranges therein and there between, by weight, based on the total weight of the composition. One of ordinary skill in the art, however, will appreciate that other ranges are within the scope of the invention. Thus, water is present, by weight, based on the total weight of the composition, from about 5, 10, 15, 20, 25, 30, 35, 40, 45, 50, 55, 60, 65, 66, 67, 68, 69, 70, 71, 72, 73, 74, 75, 80, 85, to about 90 weight percent, including increments and all ranges and subranges therein and there between.

The water used in the cosmetic composition may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the cosmetic composition is in the range from about 3 to about 7, or from about 3 to about 5, or from about 3 to about 4, or from about 4 to about 5. Generally, the pH can be adjusted to the desired value by addition of a base or an acid (organic or inorganic). In view of the inclusion of hydroxy acids in the cosmetic composition hereof, the pH is typically adjusted using a base, which in some examples is sodium hydroxide, or using an acid, which in some examples is citric acid.

Water-Soluble Solvents

In accordance with some embodiments, the cleansing composition may include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C1-C4 alcohols), polyols, glycols, and combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and combinations thereof.

In some cases, a water-soluble solvent may be selected from the group consisting of one or more glycols, C1-4 alcohols, glycerin, and combinations thereof.

In accordance with the various embodiments total amount of the at least one water-soluble solvent, when present, may vary, is from about 0.5% to about 25%, or from about 0.5% to about 20%, or from about 1% to about 20%, or from about 1% to about 10%, or from about 2% to about 5% or any suitable combination, sub-combination, range, or sub-range thereof by weight, based on the weight of the composition. One of ordinary skill in the art, however, will appreciate that other ranges are within the scope of the invention.

Thus, any one water-soluble solvent, when present, is present, by weight, based on the total weight of the composition, from about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

Humectant

In accordance with the disclosure, one or more humectants may be present in the composition. In some embodiments, the humectant may comprise one or more of polyols, including, for example, glycerin, glycerol, glycols, such as, caprylyl glycol, butylene glycol, propylene glycol, isoprene glycol, dipropylene glycol, hexylene glycol and polyethylene glycols, monoethylene glycol, diethylene glycol, triethylene glycol, diethylene glycol, hexylene glycol, glycol ethers, such as, monopropylene, dipropylene and tripropylene glycol alkyl(C1-C4)ethers, squalane, triacetin, sugars, such as, glucose, xylitol, maltitol, sorbitol, sucrose pentaerythritol, inositol, pyrrolidone carboxylic acid, lactic acid, lithium chloride, acetamide MEA, sodium lactate, urea, dicyanamide, hyaluronic acid, aloe vera, honey, and seaweed extract.

In accordance with the various embodiments, the amount of humectant or conditioning agent present in the composition can range from about 1% to about 10%, or from about 1% to about 8%, or from about 1% to about 5%, or from about 2% to about 3%, or any suitable combination, sub-combination, range, or sub-range thereof by weight, based on the weight of the composition. One of ordinary skill in the art, however, will appreciate that other ranges are within the scope of the invention. One of ordinary skill in the art, however, will appreciate that other ranges are within the scope of the invention.

Thus, any one of or a combination of humectant, when present, may be present, by weight, based on the total weight of the composition, is from about 1, 2, 3, 4, 5, 6, 7, 8, 9, to about 10 weight percent, including increments and ranges therein and there between.

Preservatives

In accordance with the disclosure, one or more preservatives and/or antimicrobials may be present in the composition. Any preservative commonly used in cosmetic cleansing compositions is an acceptable preservative for the cleansing compositions herein, such as phenoxyethanol, members from the paraben family such as the methyl, ethyl, propyl, butyl or isobutyl parabens, 4-hydroxy benzoic acid, benzoic acid, sorbic acid, dehydroacetic acid, triclosan, benzyl alcohol, chlorophenesin, or salicylic acid, for example. At more concentrated amounts of suitable solvents for optional additives, in particular, suitable solvents for antimicrobials and preservatives, members from the paraben family may be used as a preservative.

In some embodiments, the preservative may comprise one or more of preservatives selected from the group consisting of organic acids, parabens, formaldehyde donors, phenol derivatives, quaternary ammoniums, alcohols, isothiazolinones, and combinations thereof. Preservatives having antibacterial activity are optionally present in the cleansing compositions of the present invention. Examples of organic acid preservatives include, but are not limited to, sodium benzoate, potassium sorbate, benzoic acid and dehydroacetic acid, sorbic acid, and combinations thereof. A preferred organic acid preservative system includes a mixture of sodium benzoate and potassium sorbate. Examples of paraben preservatives include, but are not limited to, alkyl para-hydroxybenzoates, wherein the alkyl radical has from 1, 2, 3, 4, 5 or 6 carbon atoms and for example, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservatives include, but are not limited to, 1,3-Dimethylol-5,5-dimethylhydantoin (DMDM hydantoin), imidazolidinyl urea, gluteraldehyde, and combinations thereof. Examples of quaternary ammonium preservatives include, but are not limited to, benzalkonium chloride, methene ammonium chloride, benzethonium chloride, and combinations thereof. Examples of alcohol preservatives include, but are not limited to, ethanol, benzyl alcohol, dichlorobenzyl alcohol, phenoxyethanol, and combinations thereof. Examples of isothiazolone preservatives include, but are not limited to, methylchloroisothiazolinone, methylisothiazolinone, and combinations thereof.

In some particular embodiments, appropriate preservatives include, but are not limited to, phenoxyethanol, Tetrasodium EDTA, Zinc Gluconate, Hydroxyacetophenone, Caprylyl glycol, Chlorphenesin, or combinations thereof.

In some embodiments, the preservative includes one or more preservatives, the one or combination present at a concentration, by weight of about 0.001% to about 5%, or alternatively about 0.05% to about 2.5% or alternatively about 0.1% to about 2.0%, based upon weight of the composition. One of ordinary skill in the art, however, will appreciate that other ranges are within the scope of the invention.

Thus, any one of or a combination of preservatives, when present, may be present, by weight, based on the total weight of the composition, is from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2, 3, 4, to about 5 weight percent, including increments and ranges therein and there between.

Optional Additives

In some embodiments, the composition includes one or more of other optional ingredients selected from the group consisting of skin care actives, humectants, conditioning agents, thickeners, viscosity adjusters, cooling agents, fillers, antimicrobials, preservatives, pH adjusters, chelating agents, and combinations thereof.

In some embodiments, a cleansing composition includes at least one additive used in the cosmetics field which does not affect the properties of the composition according to the invention, such as, fragrances, pH adjusters (citric acid, sodium chloride, lactic acid); neutralizing or pH-adjusting agents (e.g., triethylamine (TEA) and sodium hydroxide, Trisodium ethylenediamine disuccinate, and combinations thereof), other cosmetically acceptable additives, such as but not limited to, pearlescent agents, silica, and coloring materials; essential oils; fruit extracts, for example, Pyrus malus (Apple) Fruit Extract, and Aloe Barbadensis Leaf Juice Powder; conditioning agents such as C12-15 Alkyl lactate and C12-15 Alcohols; Thickeners, for example Ammonium polyacryloyldimethyl taurate, Propylene glycol and PEG-55 propylene glycol oleate; viscosity adjusters, for example, Sodium chloride and Hexylene Glycol; cooling agents such as menthol; fillers, for example Kaolin; or any combination thereof. Although the optional additives are given as examples, it will be appreciated that other optional components compatible with cosmetic applications known in the art may be used.

In some embodiments, the composition includes a blend of humectants, antimicrobials, preservatives, actives, pH adjusters and chelating agents comprising C12-15 Alkyl lactate and C12-15 Alcohols, citric acid, trisodium ethylenediamine disuccinate, phenoxyethanol, caprylyl glycol, chlorphenesin, ammonium polyacryloyldimethyl taurate, propylene glycol and PEG-55 propylene glycol oleate, and kaolin.

In accordance with the various embodiments, the amount of one or more additives, alone or in combination, present in the composition can be present in the composition according to the disclosure in a range from about 0.001% to about 20%, by weight, or from about 0.005% to about 0.01%, or from about 0.01% to about 0.1%, or from about 0.15% to about 5%, or from about 0.40% to about 4%, or from about 0.5% to about 2.5% by weight, or from about 1% to about 2%, or any suitable combination, sub-combination, range, or sub-range thereof by weight, based on the total weight of the composition. One of ordinary skill in the art, however, will appreciate that other ranges are within the scope of the invention.

Thus, any one or a combination of additives may be present, by weight, based on the total weight of the composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

Although the optional additives are given as examples, it will be appreciated that other optional components compatible with cosmetic applications known in the art may be used that are suitable. It will be appreciated by a skilled artisan that any optional additives are present only to the extent and in amounts that do not materially adversely affect the basic and novel characteristic(s) of the claimed disclosure. Thus, in some embodiments that include optional additives, such optional additives will not materially adversely affect the composition.

More generally, it will be appreciated by a skilled artisan that any solvents, humectants, preservatives or other additives are present only to the extent and in amounts that do not materially adversely affect the basic and novel characteristic(s) of the claimed invention.

### EXAMPLES

The following examples are intended to further illustrate the present disclosure. They are not intended to limit the disclosure in any way. Unless otherwise indicated, all parts are by weight.

Raw Materials

Materials used included: Cationic Polymers: Chitosan, Guar Hydroxypropyltrimonium Chloride, Syntran PC 5330 (17.3% active), Polyquaternium-47 (21% active)

Salt: Sodium Chloride, Calcium Chloride, Magnesium sulfate

### Example 1: Compositions

Exemplary embodiments of cleanser compositions used as bases for the inventive compositions are set forth in Table 1, below.

TABLE 1
Base Cleanser Composition #1, pH 5.0 (“BASE 1”)

| | Concentration |
|---|---|
| BASE 1 Components | (% Active) |
| Amphoteric Surfactants | 2.1 |
| Nonionic surfactants | 4.3 |
| Anionic surfactants | 10.14 |
| Conditioning agents - Moisturizer | 3 |
| Preservatives | 0.37 |
| pH adjuster, Salts | 0.47 |
| Acne Treatment Active(s) | 2.05 |
| (including Salicylic Acid) | |
| Viscosity Adjuster | 1 |
| Cooling Agent | 0.1 |
| Water | q.s. to 80 |

TABLE 2
Base Cleanser Composition #2, pH 5.0 (“BASE 2”)

| | Concentration |
|---|---|
| Component | (% Active) |
| Amphoteric Surfactants | 7.5 |
| Nonionic surfactants | 1 |
| Anionic surfactants | 5.4 |
| Conditioning agents - Moisturizer | 5 |
| Preservatives | 0.8 |
| pH adjuster, Salts | 0.4 |
| Acne Treatment Active(s) | 2 |
| (including Salicylic Acid) | |
| Water | q.s. to 80 |

TABLE 3
Base Cleanser Composition #3, pH 4.0 (“BASE 3”)

| BASE 3 | Concentration |
|---|---|
| Component | (% Active) |
| Amphoteric Surfactants | 5.4 |
| Nonionic surfactants | 2.5 |
| Anionic surfactants | 4.4 |
| Conditioning agents - Emollient | 1.5 |
| Preservatives | 1.1 |
| pH adjuster, Salts | 0.6 |
| Chelating agent | 0.19 |
| Thickeners | 1.6 |
| Filler | 2 |
| Acne Treatment Active(s) | 2 |
| (including Salicylic Acid) | |
| Water | q.s. to 80 |

Inventive Compositions:

An Exemplary Inventive Composition is shown below in Table 4. All concentrations shown in Table 4 are final percentages of actives based on the weight of the composition, wherein each is based on use of raw materials with active percentages that are essentially 100% except as indicated: * 17.3% active; ** 21% active

TABLE 4
Exemplary Inventive Compositions

| | | | | | Cleanser | Cleanser | Cleanser | Cleanser |
|---|---|---|---|---|---|---|---|---|
| | with | with | with | with | with | with | with | with PQ- |
| Component | Syntran | Chitosan | Guar | PQ-47 | Syntran | Chitosan | Guar | 47 |
| Amphoteric | optional | optional | optional | optional | about | about | about | about |
| Surfactant(s) | | | | | 1.0% to | 1.0% to | 1.0% to | 1.0% to |
| | | | | | about | about | about | about |
| | | | | | 10% | 10% | 10% | 10% |
| Anionic | optional | optional | optional | optional | about | about | about | about |
| Surfactant(s) | | | | | 1.0% to | 1.0% to | 1.0% to | 1.0% to |
| | | | | | about | about | about | about |
| | | | | | 10% | 10% | 10% | 10% |
| Nonionic | optional | optional | optional | optional | about | about | about | about |
| Surfactant(s) | | | | | 1.0% to | 1.0% to | 1.0% to | 1.0% to |
| | | | | | about | about | about | about |
| | | | | | 10% | 10% | 10% | 10% |
| Thickener(s)/ | optional | optional | optional | optional | about | about | about | about |
| Viscosity | | | | | 0.15% to | 0.15% to | 0.15% to | 0.15% to |
| Adjuster(s)/ | | | | | about | about 5% | about | about |
| Filler(s) | | | | | 5% | | 5% | 5% |
| Conditioning | optional | optional | optional | optional | about | about | about | about |
| Agent(s) | | | | | 0.5% to | 0.5% to | 0.5% to | 0.5% to |
| | | | | | about | about | about | about |
| | | | | | 10% | 10% | 10% | 10% |
| Syntran PC 5330* | about | — | — | — | about | — | — | — |
| | 0.01% to | | | | 0.01% to | | | |
| | about | | | | about | | | |
| | 0.5% | | | | 0.5% | | | |
| Chitosan | — | about | — | — | — | about | — | — |
| | | 0.01% to | | | | 0.01% to | | |
| | | about | | | | about | | |
| | | 0.5% | | | | 0.5% | | |
| Cationic Guar | — | — | about | — | — | — | about | — |
| | | | 0.01% | | | | 0.01% to | |
| | | | to | | | | about | |
| | | | about | | | | 0.5% | |
| | | | 0.5% | | | | | |
| Polyquaternium- | — | | — | about | — | | — | about |
| 47** | | | | 0.01% | | | | 0.01% to |
| | | | | to | | | | about |
| | | | | about | | | | 0.5% |
| | | | | 0.5% | | | | |
| Water | QS | QS | QS | QS | QS | QS | QS | QS |

Embodiments of the inventive cleanser composition were prepared using one the exemplified base compositions and are further described herein below. It will be appreciated that the exemplified base compositions are not limiting, and that yet other embodiments of the inventive cleanser composition may be prepared in accordance with the scope of the disclosure and may include the same or other ingredients that do not materially adversely affect the basic and novel characteristics of the claimed invention.

Inventive Compositions with BASE 3

Exemplified Inventive Compositions were prepared with the exemplified BASE 3 plus a cationic polymer, as shown in these examples, chosen from Syntran PC 533* (“Syntran”), Chitosan, Cationic Guar (“Guar”), and Polyquaternium 47** (“PQ-47”). Specific examples of Inventive Compositions that include one of these exemplified cationic polymers are shown below in Table 5.

TABLE 5
Inventive Compositions with BASE 3

| | INVENTIVE 1 | INVENTIVE 2 | INVENTIVE 3 | | INVENTIVE 4 | | INVENTIVE 5 |
|---|---|---|---|---|---|---|---|
| | BASE 3 + | BASE 3 + | BASE 3 + | | BASE 3 + | | BASE 3 + |
| | water | Syntran | Chitosan | | Guar | | PQ-47 |
| BASE 3 | 80 g | 80 g | 80 | g | 80 | g | 80 g |
| Syntran PC 5330* | — | 1 g | — | | — | | — |
| Chitosan | — | — | 0.2 | g | — | | — |
| Cationic Guar | — | — | — | | 0.2 | g | — |
| Polyquaternium-47** | — | — | | | — | | 1 g |
| Water | 20 g | 19 g | 19.8 | g | 19.8 | g | 19 g |

*17.3% active;
**21% active

Inventive Compositions with BASE 1 and BASE 2:

Inventive Compositions were prepared with the bases BASE 1 and BASE 2, +a cationic polymer, as shown in these examples, chosen from Syntran, Chitosan, Cationic Guar, and Polyquaternium-47. The Example Compositions are shown in Table 6.

TABLE 6
Inventive Compositions with one of Base 1 or Base 2

| | INV 6 | INV 7 | INV 8 | INV 9 | INV 10 | INV 11 | INV 12 | INV 13 |
|---|---|---|---|---|---|---|---|---|
| | BASE 1 + | BASE 1 + | BASE 1 + | BASE 1 + | BASE 2 + | BASE 2 + | BASE 2 + | BASE 2 + |
| | Syntran | Chitosan | Guar | PQ-47 | Syntran | Chitosan | Guar | PQ-47 |
| BASE 1 | 80 g | 80 g | 80 g | 80 g | | | | |
| BASE 2 | | | | | 80 g | 80 g | 80 g | 80 g |
| Syntran* | 1 g | — | — | — | 1 g | — | — | — |
| Chitosan | — | 0.2 g | — | — | — | 0.2 g | — | — |
| Guar | — | — | 0.2 g | — | — | — | 0.2 g | — |
| PQ-47** | — | — | — | 1 g | — | | — | 1 g |
| Water | 19 g | 19.8 g | 19.8 g | 19 g | 19 g | 19.8 g | 19.8 g | 19 g |

*17.3% active; **21% active

Comparative Compositions

Comparative Compositions include the above listed BASE 1 and BASE 2 compositions and were prepared with addition of water. The Comparative Compositions and are shown below in Table 7.

TABLE 7
Comparative Compositions

| | COMP 1 | COMP 2 | COMP 3 |
|---|---|---|---|
| BASE 1 | 80 g | | |
| BASE 2 | | 80 g | |
| BASE 3 | | | 80 g |
| Syntran PC 5330* | — | — | — |
| Chitosan | — | — | — |
| Cationic Guar | — | — | — |
| Polyquaternium-47** | — | — | — |
| Water | 20 g | 20 g | 20 g |

*17.3% active;
**21% active

### Example 2. Demonstration of Salicylic Acid Deposition

Compositions were evaluated for demonstration of salicylic acid deposition on a synthetic skin material after application of the various tested compositions. The test set up is shown below.

Salicylic Acid Deposition Test

Processing of BIOSKIN Samples: BIOSKIN samples of about 2 in by 2 inches were provides. Each bioskin sample was treated with one of an inventive, example and comparative composition by application of about 0.5 g of the tested composition with a cleansing brush (Clarisonic™ brush). Cleansed samples were soaked in water for about 30 seconds, then massaged using the cleansing brush for about one minute. The samples were then rinsed under water at forty degrees C. then air dried for about an hour. Samples were then soaked in a solution of iron chloride (0.01% FeCl₃) for about an hour then air dried overnight.

The color of each sample of treated BIOSKIN (vs a control sample lacking any treatment) was measured using a hand-held colorimeter and the measurements were analyzed to provide a color change (resulting from binding to the iron compound to deposited SA) reflected as a percentage change in color relative to the control. The results are shown Tables 8-10.

TABLE 8
SALICYLIC ACID DEPOSITION WITH INVENTIVE COMPOSITIONS

| | COMP 3 | INVENTIVE 2 | INVENTIVE 3 | INVENTIVE 4 | INVENTIVE 5 |
|---|---|---|---|---|---|
| | BASE 3 + | BASE 3 + | BASE 3 + | BASE 3 + | BASE 3 + |
| | water | Syntran | Chitosan | Guar | PQ-47 |
| % CH A | 438.15 | 626.9 | 936.72 | 555.7 | 577.61 |

From the data above, addition of all the cationic polymers promoted the salicylic acid deposition for each of the Inventive Compositions over the Comparative composition (Base plus water with no cationic polymer), as indicated by the change of the A value.

TABLE 9
SALICYLIC ACID DEPOSITION OF COMPARATIVE 1
AND INVENTIVE COMPOSITIONS INCLUDING BASE 1

| | COMP 1 | INV 6 | INV 7 | INV 8 | INV 9 |
|---|---|---|---|---|---|
| | BASE 1 + | COMP 1 + | COMP 1 + | COMP 1 + | COMP 1 + |
| | water | Syntran | Chitosan | Guar | PQ-47 |
| % CH A | 177.05 | 271.57 | 277.26 | 236.6 | 189.55 |

From the data above, addition of all the cationic polymers promoted the salicylic acid deposition for each of the Inventive Compositions over the Comparative composition (Base plus water with no cationic polymer), as indicated by the change of the A value.

TABLE 10
SALICYLIC ACID DEPOSITION COMPARATIVE 2 AND
INVENTIVE COMPOSITIONS INCLUDING BASE 2

| | COMP 2 | INV 10 | INV 11 | INV 12 | INV 13 |
|---|---|---|---|---|---|
| | BASE 2 + | COMP 2 + | COMP 2 + | COMP 2 + | COMP 2 + |
| | water | Syntran | Chitosan | Guar | PQ-47 |
| % CH A | 111.1 | 145.14 | 134.14 | 181.66 | 175.58 |

From the data above, addition of all the cationic polymers promoted the salicylic acid deposition for each of the Inventive Compositions over the Comparative composition (Base plus water with no cationic polymer), as indicated by the change of the A value.

Referring to Tables 8-10, in all instances, Comparative compositions that did not include any cationic polymer demonstrated the lowest amount of post-rinse deposited salicylic acid. In contrast, each of the Inventive compositions that included one of the exemplified cationic polymers selected from Syntran, Chitosan, Cationic Guar, and Polyquaternium-47 demonstrated significantly increased post-rinse deposited salicylic acid. While these results are obtained with the specifically exemplified cationic polymers, the invention includes other embodiments of cationic polymers and combinations thereof according to the disclosure that would be contemplated to provide the same or comparable deposition of salicylic acid.

Generally, embodiments of the composition according to the disclosure confer deposition and retention of the beta hydroxy acid, after rinsing, in a range from about 50% to about 250% greater, or more, than the deposition and retention, after rinsing, that is obtained by a similar composition that lacks a cationic polymer. Thus, the relative increase in deposition of beta hydroxy acid, for example, salicylic acid, obtained using an inventive composition containing a cationic polymer is greater than what is obtained in the absence of cationic polymer in a range from about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, to about 250 percent, including increments and ranges therein and there between.

The retention of salicylic acid on the skin even beyond cleansing with our without a cleansing brush would extend the duration of the salicylic acid effectiveness for addressing acne and other skin conditions that are responsive to salicylic acid and other beta hydroxy acids, thereby providing sustained treatment in an otherwise rinsible formulation.

### Example 3. Demonstration of the Effect of Salt on Salicylic Acid Deposition

Compositions were evaluated for demonstration of the effect of salt on salicylic acid deposition on a synthetic skin material after application of the various tested compositions on the BIOSKIN. The tested composition is shown in Table 11 and results of the effect of added salt on deposition are shown in Table 12.

TABLE 11
Inventive Formulation tested for effect of added salt

| | INV 10 |
|---|---|
| | BASE 2 + |
| | Syntran |
| BASE 2 | 80 g |
| Syntran* | 1 g |
| Water | 19 g |

*17.3% active

TABLE 12
PERCENT SALICYLIC ACID COVERAGE WITHOUT
AND WITH ADDITION OF VARIOUS SALTS

| | INVENTIVE | INVENTIVE | INVENTIVE | INVENTIVE |
|---|---|---|---|---|
| | 10 | 10 + NaCl | 10 + CaCl₂ | 10 + MgSO₄ |
| % Coverage | 38 | 72 | 62 | 70 |
| of SA | | | | |

That data obtained using BASE 2 with the added cationic polymer Syntran with and without one of each of NaCl, CaCl2, MgSO4 at 0.2% are shown in Table 12. The data show that salt added into the composition provides enhanced deposition of salicylic acid. It is posited that the additional salt helps facilitates precipitation of the salicylic acid to enhance retained deposition on the bioskin during rinse off. Thus, embodiments according to the disclosure that include supplemental salt can provide further enhanced deposition of actives for extend the duration of the salicylic acid effectiveness for addressing acne and other skin conditions that are responsive to salicylic acid and other beta hydroxy acids, thereby providing sustained treatment in an otherwise rinsible formulation.

While the disclosure has been described with reference to described embodiments, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one” or “one or more” as used herein, means that there may be one, two, three or more and thus includes individual components as well as mixtures/combinations.

The transitional terms “comprising,” “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the cosmetic composition, typically 0% by weight, based on the total weight of the cosmetic composition. The term “essentially free” means that, while it prefers that no excluded material is present in the cosmetic composition, it is possible to have very small amounts of the excluded material in the cosmetic composition of the invention, provided that these amounts do not materially affect the advantageous properties of the cosmetic composition. In particular, “essentially free” means that excluded material can be present in the cosmetic composition at an amount of less than about 0.1% by weight, based on the total weight of the cosmetic composition.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated. As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc. As used herein a range of ratios is meant to include every specific ratio within, and combination of subranges between the given ranges.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic composition comprising:
(a) at least one acne treatment active comprising at least one beta hydroxy acid; and
(b) an aqueous carrier comprising at least one cationic polymer selected from the group consisting of chitosan, cationic guar, an amorphous functional acrylic polymer grafted onto a polyethylene backbone, a polyquaternium, and combinations thereof,
wherein the composition when applied to a keratinous tissue demonstrates greater deposition and retention of the at least one beta hydroxy acid on the keratinous tissue after rinsing with water in an amount representing about 60% to about 250% as compared with an amount deposited with a composition that lacks the at least one cationic polymer.

2. The cosmetic composition according to claim 1, wherein the deposition and enhanced beta hydroxy acid retention is demonstrated by measurement of color change resulting from complex formation between the at least one beta hydroxy acid and a metal.

3. The cosmetic composition according to claim 1, wherein the deposition of beta hydroxy acid demonstrated is stable and resistant from removal by rinsing or use of a cleansing apparatus.

4. The cosmetic composition according to claim 1, wherein the at least one beta hydroxy acid is selected from the group consisting of -n-octanoylsalicylic acid, salicylate, sodium salicylate, capryloyl salicylic acid, willow extract, beta hydroxybutanoic acid, tropic acid, trethocanic acid, and combinations thereof.

5. The cosmetic composition according to claim 1, wherein the at least one beta hydroxy acid comprises salicylic acid.

6. The cosmetic composition according to claim 1, wherein the at least one beta hydroxy acid is present in the composition in a range from about 0.1% to about 2%, by weight, based on the total weight of the composition.

7. The cosmetic composition according to claim 1, wherein each of the at least one cationic polymer is present in an amount that ranges from about 0.01% to about 0.5% by weight based on the total weight of the composition.

8. The cosmetic composition according to claim 1, further comprising at least one salt present in the composition in a range from about 0.01% to about 2%, by weight, based on the total weight of the composition.

9. The cosmetic composition according to claim 1, further comprising at least one salt selected from the group consisting of sodium chloride, calcium chloride, potassium chloride, magnesium sulfate, magnesium carbonate, calcium carbonate, zinc pyrrolidone carboxylic acid, and combinations thereof.

10. (canceled)

11. The cosmetic composition according to claim 1, wherein the at least one cationic polymer is selected from the group consisting of chitosan, cationic guar, and combinations thereof.

12. (canceled)

13. The cosmetic composition according to claim 1, wherein the at least one cationic polymer is selected from the group consisting of chitosan, cationic guar, an amorphous functional acrylic polymer grafted onto a polyethylene backbone, polyquaternium-47, and combinations thereof.

14. The cosmetic composition according to claim 1, further comprising at least one surfactant present in an amount that ranges from about 0.1% to about 20% by weight based on the total weight of the composition.

15. The cosmetic composition according to claim 1, further comprising at least one surfactant selected from the group consisting of nonionic surfactants, cationic surfactants, anionic surfactants, amphoteric surfactants, and combinations thereof.

16. The cosmetic composition according to claim 1, further comprising a combination of amphoteric, nonionic, and anionic surfactants comprising coco betaine, glyceryl stearate, PEG-150 Distearate, and sodium methyl cocoyl taurate.

17. The cosmetic composition according to claim 1, further comprising one or more components selected from the group consisting of other skin care actives, humectants, conditioning agents, thickeners, viscosity adjusters, cooling agents, fillers, antimicrobials, preservatives, pH adjusters, chelating agents, and combinations thereof.

18. The cosmetic composition according to claim 1, wherein the composition is a cleanser.

19. A cosmetic composition comprising:
(a) at least one acne treatment active comprising at least one beta hydroxy acid present in the composition in a range from about 0.1% to about 2%, by weight, based on the total weight of the composition; and
(b) an aqueous carrier comprising:
i. at least one cationic polymer selected from the group consisting of chitosan, cationic guar, an amorphous functional acrylic polymer grafted onto a polyethylene backbone, a polyquaternium, and combinations thereof and present in an amount that ranges from about 0.01% to about 0.5% by weight based on the total weight of the composition,
ii. at least one salt present in the composition in a range from about 0.01% to about 2%, by weight, based on the total weight of the composition;
iii. at least one surfactant present in an amount that ranges from about 0.1% to about 20% by weight based on the total weight of the composition,
wherein the composition is a skin cleanser, and
wherein the composition when applied to a keratinous tissue demonstrates greater deposition and retention of the at least one beta hydroxy acid on the keratinous tissue after rinsing with water in an amount representing about 60% to about 250% as compared with an amount deposited with a composition that lacks the at least one cationic polymer.

20. A cosmetic composition comprising:
(a) at least one acne treatment active comprising at least one beta hydroxy acid that comprises salicylic acid; and
(b) an aqueous carrier comprising:
i. at least one cationic polymer selected from the group consisting of chitosan, cationic guar, an amorphous functional acrylic polymer grafted onto a polyethylene backbone, polyquaternium-47, cationic polyurethanes, and combinations thereof,
ii. at least one salt selected from the group consisting of sodium chloride, calcium chloride, magnesium sulfate potassium chloride, zinc pyrrolidone carboxylic acid, and combinations thereof,
iii. at least one surfactant selected from the group consisting of nonionic surfactants, cationic surfactants, anionic surfactants, amphoteric surfactants, and combinations thereof,
wherein the composition is a skin cleanser, and
wherein the composition when applied to a keratinous tissue demonstrates greater deposition and retention of the at least one beta hydroxy acid on the keratinous tissue after rinsing with water in an amount representing about 60% to about 250% as compared with an amount deposited with a composition that lacks the at least one cationic polymer.
